(19) United States
(12) Patent Application Publication
(10) Pub. No.: US 2002/0139419 A1
(43) Pub. Date: Oct. 3, 2002

(54) Programmable flexible-tube flow regulator and use methods

(76) Inventor: David E. Flinchbaugh, Orlando, FL (US)

Correspondence Address: LAW OFFICES OF BRIAN S STEINBERGER, 101 BREVARD AVENUE, COCOA, FL, 32922, US

(21) Appl. No.: 10/114,792

(22) Filed: Apr. 2, 2002

Related U.S. Application Data
(63) Continuation-in-part of application No. 10/010,534, filed on Dec. 7, 2001.
(60) Provisional application No. 60/281,056, filed on Apr. 2, 2001.
(60) Provisional application No. 60/280,767, filed on Apr. 2, 2001.
(60) Provisional application No. 60/280,768, filed on Apr. 2, 2001.
(60) Provisional application No. 60/324,601, filed on Sep. 25, 2001.

(51) Int. Cl. A61M 39/28; F16K 37/00

(52) U.S. Cl. 137/557; 604/65; 604/34; 604/250; 137/485

(57) ABSTRACT

Programmable methods and apparatus for providing flow control in valves. In a preferred embodiment flow control occurs by pressing opposite sides of a very flexible tube section together to selectively, and programmably, closing and opening the flow pathway. Linear or rotary cam electrically motorized members provide valve function without requiring fluid flow through the valve. Alternatively, micro-fluidic amplifiers and fluidic logic circuits can be used to sense and control fluid pressure in a pre-calculated proportional flow regulation pattern. The invention can be used in medical, hospital, clinic and home-care units for bladder cycling a patient whose urinary tracts have an indwelling catheter, utilizing a pressure transducer that opens and closes the valve in accordance with preprogrammed pressure buildup or time intervals. Other uses include a medical infusion subsystem by regulating flow of fluid from a medication reservoir into the blood or into a digestive system. Electromechanical and electrochemical sensors coupled by tubing to reservoirs can collect electromechanical data. Additional uses can include industrial and consumer applications can include kidney dialysis, heart catheters, and neutral or cranial stents, and the like, can be additional medical areas for specific computer-chip controlled flow regulation units. Displays and/or parameter warning indicators can also be used.

This invention relates to hospital, clinical or home-care medical instruments, and in particular to a programmable, pressure transducer-controlled bladder-drainage cycler, and programmable, electrochemical-transducer-controlled medication infusion system that can be made to be implantable or operable from outside the body of a patient, for use as flow regulators for regulating flow of fluid through a flexible tube for various industrial and consumer applications, and this invention claims the benefit of priority to U.S. Provisional Application 60/281,056 filed Apr. 2, 2001, and this invention is a Continuation-In-Part of U.S. application Ser. No. 10/010,534 filed Dec. 7, 2001, which claims the benefit of priority to U.S. Provisional Application 60/280,767 filed Apr. 2, 2001 and U.S. Provisional Application 280,768 filed Apr. 2, 2001 and U.S. Provisional Application 60/324,601 filed Sep. 25, 2001.

### BACKGROUND AND PRIOR ART

Hospital instruments and procedures for draining bladders of patients have evolved from constant uncycled drainage through siphoning, suction and various types of cyclic methods. Fundamental to an effective system is allowing the bladder to fill reasonably and then draining it without a suction effect and without allowing build-up or entry of infectious contaminants in the drainage system. Aspects to be avoided in bladder cycling are constant(continuous)drainage, excessive negative pressure and incomplete drainage of either the bladder or the catheter equipment being used.

U.S. Pat. Nos. 4,856,588 and 5,114,412 by Flinchbaugh, the same inventor of the subject invention describes “Magnetic Bladder Cycler . . . ”, title, that provided greater accuracy and reliability in filling the needs of an effective bladder cycler in comparison to prior art devices. However, these devices were not fully programmable for automated use. Nor did these devices permit the control of fluids through a medical tube without flow also through the control mechanism. Flow of fluid through the control mechanism required the use of a new mechanism for each new patient and periodically, a new mechanism for the same patient to minimize contamination.

However, this invention allows repeated use of the same control mechanism for different patients. Only the medical tubing needs changing to avoid contamination. The control unit can have a long use-life at low cost for a medical tube system without contamination. Further, it is programmable for total control to meet the needs of each patient completely.

For infusion of medication into the body of patients, there is currently a programmable medication infusion system which is implantable in the body as described in U.S. Pat. No. 4,373,527. However, this system controls flow through valves and control mechanism components rather than through a tube.

In addition to controlling infusion of medication more accurately and reliably, flow control through a tube decreases contamination by avoiding the many crevices for growth of contamination in and around the components of control mechanisms. Highly significant also, the tube can be replaced easily at minimal costs in comparison to difficult and expensive replacement of present programmable medication infusion mechanisms when they have become inoperable or contaminated.

Additionally, this invention does not require changing as often because it avoids contamination for which changing would be required and its components are longer-lasting and more reliable.

Long use-life, high reliability and greater accuracy of this invention for medication infusion and for bladder cycling results from simplicity of its components. Medication can be supplied in either a continuous small stream or in doses as required. Requirements for medication can be determined by electrochemical transducers to assure proper dosage and to prevent overdose hazards. Bladders can be cycled completely without hazardous suction. The medical tubing used can be collapsible to avoid contamination.

A major feature of this invention is that in all medical uses, its fluid flow-control mechanism is most nearly like the natural flow control of fluids by muscular contraction and expansion in a living body. The invention is more appropriate and compatible for use by the body.

The type of programming to which this invention is suited for most purposes is basically analog as contrasted to digital programming. This approximates body functions as well as most requirements of flow regulation.

Smallness in size is critical for implantation of medication infusion systems. This invention can be made much smaller than present control systems because it is simpler in construction and there are fewer parts.

Included in previous devices for bladder cycling have been U.S. Pat. Nos. 2,602,448 and 2,860,636 which utilized a siphon in combination with a reservoir to provide cyclic draining of the bladder. Pressure release in these devices are controlled by raising the height of the device on a bedside tree. These devices are subject to distortion by shifting and turning of the patient and, therefore, very undependable in addition to being restrictive of the patient.

In U.S. Pat. No. 3,598,124, a siphon leg is controlled by merely attaching a catheter to a bedside tree at predetermined adjusted height, which varies the pressure at which the bladder will drain and provides a flutter valve near the patient to break the siphon action of the system once the bladder has drained. In U.S. Pat. No. 4,230,102, a device for the draining of a bladder is shown in which a T-joint has been placed on a catheter and has a pressure membrane attached thereto in a large casing for actuating a pressure switch which in turn actuates an electric motor driving a gear train and cam. A cam follower is spring loaded to close the catheter for two-minute cycles upon actuation by the pressure switch to drain the bladder. This type of device, however, is expensive and bulky, and requires fluid to pass external to the catheter to be measured. This device would require cleaning after being used since fluid physically contacts it.

In U.S. Pat. No. 4,424,058, a spring-return valve is provided in conjunction with a siphon release orifice to prevent excessive suction and to prevent urine from remaining in the system after drainage. A problem with this system was that resistance of the spring increased with distance of travel from a closed position. This tended to cause some fluid to remain in the bladder because only a full bladder would open it and only a relatively full bladder would keep it open sufficiently to allow complete drainage unless overridden by the patient. Also, positioning of tubes leading from it were parallel to the leg on which it was attached and provided a situation for retention of fluid in the system.

### SUMMARY OF THE INVENTION

The first objective of the invention is to provide for repeated use of the same control mechanism for different patients, with only the medical tubing needing to be changed to avoid contamination. The control unit can have a long use-life at low cost for a medical tube system without contamination.

The second objective of the invention is to provide a programmable flow regulator for regulating flow of fluid through flexible tubes for total control to meet the needs of each patient completely. The controls can include a programmable, pressure transducer-controlled bladder-drainage cycler, and programmable, electrochemical-transducer-controlled medication infusion system that can be made to be implantable or operable from outside the body of a patient.

A third objective of the invention is to provide a programmable flow regulator for longitudinal tubes that can attach about sides of tube with fluid flowing therethrough, without having to separate, cut, or remount the tube. The invention can be easily attached to any tube in which fluid flows therethrough, and unattached without having to spill any fluid flowing through the tube.

The fourth objective of the invention is to provide a programmable flow regulator for fluid flowing through tubes having capabilities for displaying operating parameters and status parameters of the regulator, and/or system warning indicators and/or transceiver and receiver capabilities for sending and/or receiving data from remote stations.

In this invention, flow of fluid through flexible tube is stopped, allowed or regulated in amount by programmable pressing of the sides of the flexible tube together selectively. For hospital and other medical applications, the flexible tube can be a catheter tube for cycling a bladder or an infusion tube for infusing medication into the blood stream or for infusing medication into the digestive tract. The flow through such tubes can be programmable for control by a pressure transducer, by a chemical transducer by an electrochemical transducer or by a timer. Either can be made to be overridden manually.

Applications of this invention for cycling bladders and for infusing medication differ primarily in whether input or output of fluid is being controlled. The types of flexible tubing utilized and the types of transducers utilized for programmable control also can be different. When used as a bladder cycler, the inlet tube utilized is a catheter because the source, or fluid is the bladder. When used as a medication infuser, the inlet tube is a medication tube because the source of fluid is a medication reservoir. The outlet tube is an excretion discharge conveyance when this invention is employed as a bladder cycler. When employed as a medication infuser, the outlet tube is a medication infusion tube that is attachable selectively to either the blood system or the digestive tract of a patient.

The programmable control principles and mechanisms for both applications are substantially the same except that flow regulation is controlled programmably by a pressure transducer for a bladder cycler and by electrochemical transducers for medication infusion. Both are programmable also for timed valve closing, opening and variable flow regulation. Also, both are provided with manual override.

With appropriate modification but with substantially the same basic principles, the medication infuser can be miniaturized for implantation into the body of the patient. Partly because it is simple and yet basically free from contamination in operation, it can be made smaller and more reliable than other implantable, programmable medication infusion systems.

In this invention, a controllable-step motor, preferably a linear motor, is utilized to actuate a rod against a flexible tube supported by a base member at the opposite side of the tube in order to prevent or to selectively allow flow or fluid through the tube. When used as a bladder cycler, a pressure sensor at an inlet side of the rod and base member senses radially outward pressure in the tube and actuates a switch to activate the linear motor as programmed. When used as a medication infuser, either a chemical transducer, an electrical transducer or both can be employed. Fluid flow or medication through infusion tubes to the blood system or to the digestive tract of patients can be programmed as required.

The controllable-step motor can also be rotational with a gear drive of a rod which functions as a valve to open and close the tube for selective regulation or flow of fluid. A variety of mechanisms are described for programmable flow control through a flexible tube with this invention.

When used for various industrial add consumer applications, the size of the components can be adjusted to the particular needs.

Miniaturization of the invention for such applications as implantable, programmable medication infusion systems can be aided by the use of collapsible tubes to decrease the energy required to operate the linear valve and thereby to decrease the size or the unit.

Collapsible tubes also decrease contamination by decreasing fluid remaining within the tubes when not otherwise conveying fluid. This further decreases stagnation conditions in the system.

Further objects and advantages of this invention will be apparent from the following detailed description of a presently preferred embodiment which is illustrated schematically in the accompanying drawings.

### BRIEF DESCRIPTION OF THE FIGURES

FIG. 1 is a cutaway side view of a bladder-cycler embodiment of the invention employing. a linear motor in direct actuation of a flexible-tube flow regulator.

FIG. 2 is a top view of the embodiment illustrated in FIG. 1.

FIG. 3 is a top view of a medication-infusion embodiment of the invention utilizing a linear motor similarly to the FIG. 1 illustration.

FIG. 4 is a schematic side view with a rotational electrically-motorized member in conjunction with an inclined plane.

FIG. 5 is a schematic side view with a linear electrically-motorized member in conjunction with an inclined plane.

FIG. 6 is a schematic side view with a linear electrically-motorized member to actuate a pliers-like lever and fulcrum.

FIG. 7 is a flow diagram of a bladder-cycler embodiment of the invention.

FIG. 8 is a flow diagram of a medication-infusion embodiment of the invention.

FIG. 9 is a circuit diagram of the invention illustrating circuitry for bladder-cycler, medication-infusion and other applications.

### DESCRIPTION OF THE PREFERRED EMBODIMENTS

Before explaining the disclosed embodiments of the present invention in detail it is to be understood that the invention is not limited in its application to the details of the particular arrangement shown since the invention is capable of other embodiments. Also, the terminology used herein is for the purpose of description and not of limitation.

This invention is a Continuation-In-Part of U.S. application Ser. No. 10/010,534 filed Dec. 7, 2001, whose subject matter is incorporated by reference.

Referring to FIG. 1, a contractible member is actuated selectively in the direction of or in the opposite direction from a base member 2 to regulate flow or fluid through a flexible tube 3. Motion for actuation of the contractible member 1 is transferred through motive member 4 from plunger 5 which can be a conductor in a linear motor having electrical coils 6 in a linear electrical step motor 7. The linear step motor can also be a rotational motor, preferably a rotational step motor, that operates gears. Because either a linear motor or a rotational motor with gears can be employed, an electrical motor of either type is referred to alternatively in this description as an electromotive means. Contractible member 1 and base member 2 can be referred to together as a regulator valve which is opened fully or partially by movement of contractible member 1 selectively in a direction away from base member 2. It is closed by movement of contractible member 1 in a direction towards base member 2 to press opposite sides of flexible tube 3 together with sufficient force to prevent flow of fluid through the tube 3.

Electrical current is provided to the electrical coils 6 by battery 8 which can be chargeable. Electrical current for charging battery 8 is supplied from battery charge box 9 having electrical plug 10. Low voltage current is transmitted through charger wire 11 to converter 12. The converter 12 can be a converter of relatively high voltage to relatively lower voltage within a low-voltage system. The battery charge box 9 is employed to isolate high current or an electrical source from low current in association with a patient or other use condition. This prevents electrical hazards in addition to providing efficient charging of a battery.

An optional tube cover 13 is swivelable on hinge bolt 14 to prevent the flexible tube 3 from escaping from between the contractible member 1 and the base member 2 under use conditions.

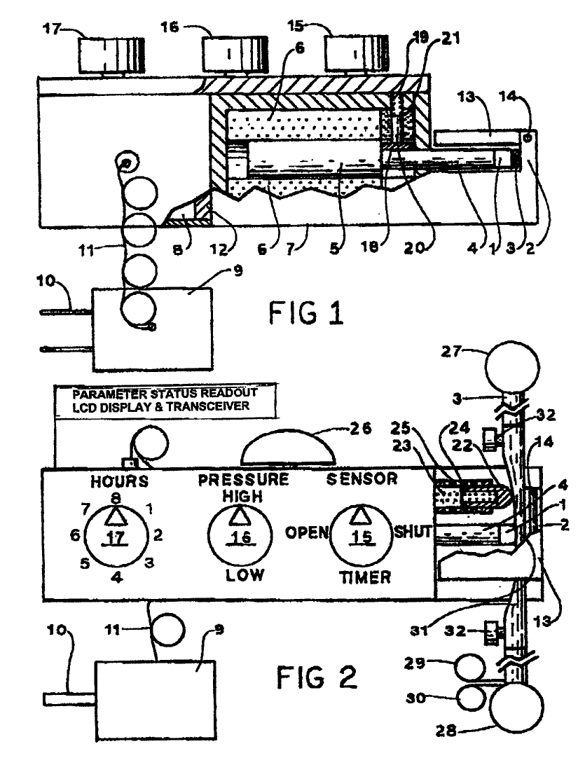

Referring now to both FIG. 1 and FIG. 2, a regulator knob 15 is rotatable in either direction to select the programmable regime. At the position designated “SENSOR” at a 12 O'clock position, the program selected is for closing of contractible member 1 and opening it in accordance with pressure in flexible tube 3. The amount of pressure required to open the regulator valve by movement of contractible member 1 away from base member 2 and flexible tube 3 is determined by rotation of pressure knob 16 to require a relatively high pressure at the “HIGH” position at 12 O'clock or a relatively low pressure at the “LOW” position at 6 O'clock, markings. The knobs are operated by rotating the arrow on each knob to a desired rotational setting.

The regulator valve is closed and set on timed opening rather than pressure-sensor opening by turning regulator knob 15 to “TIMED” position at 6 O'clock on the dial. Time interval for opening the regulator valve by moving contractible member 1 away from base member 2 is determined by rotation of timer knob 17 to hours and portions of hours indicated clockwise on a dial around the outside periphery of timer knob 17.

Referring back to FIG. 1, the motive member 4 is held mechanically by brake shoe 18 in whatever position it is moved by the plunger 5. Inward travel of the brake shoe 18 for braking effect is caused by biased pressure or brake spring 19 against brake plunger 20. Brake-releasing outward travel of the brake shoe 18 is caused by electrical charging of electrical brake coil 21. The electrically-motorized means to actuate release of brake shoe 18 can be a linear motor in which electrical current is directed to brake coil 21 from electrical current circuited to electrical coils 6. This causes the brake plunger 20 to travel outwardly to release the brake regardless or which direction the valve plunger 5 is caused to travel. Thus, the valve plunger 5 is released to actuate travel of motive member 4 in either direction but held precisely in programmed positions without expenditure or electrical current when not actuated electrically.

There can be a series of brake shoes 18 and brake coils 21 circumferentially around the outside periphery of plunger 5. The brake shoes and the surface of the plunger can be made appropriate in size and physical nature to maximize effectiveness of this braking system. The surfaces of multiple brake shoes 18 and the surface of motive member 4 can be appropriately toothed or roughened and staggered for the brake shoes 18 to hold the motive member 4 precisely and reliably with only minimal tension and size of brake spring 19.

Low tension of brake spring 19 is desirable to minimize the current required to release the brake shoe 18 for travel of the motive member 4. This reduces the size of brake coil 21 as well as the size and amount of current required for electrical coil 6.

Referring to FIG. 2. a pressure-sensing member 22 is biased against flexible tube 3 by pressure-transducer resilient member 23. Increase of pressure in flexible tube 3 actuates the outside periphery of the flexible tube 3 against the sensing member 22 and causes pressure-transducer resilient member 23 to contract and travel in a direction away from the flexible tube. This travel causes electrically-conductive transducer-plunger points 24 to contact matching stationary pressure transducer points 25. This contact can be programmed to signal opening travel of electromotive means 5.

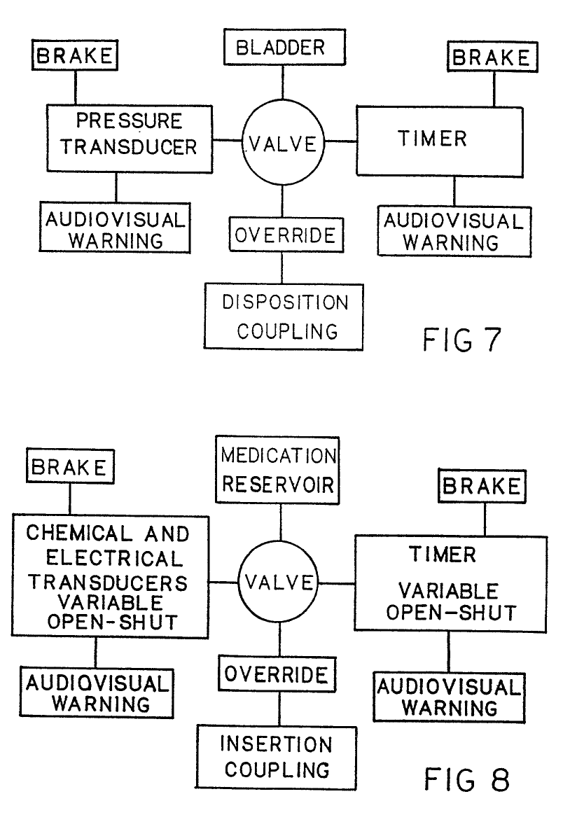

Simultaneously with opening travel of electromotive means 5, electrical current can be circuited to activate audiovisual warning means 26 which is represented by a circular bell in FIG. 2. This is designated by the words “AUDIOVISUAL WARNING” in a FIG. 7 flow diagram.

The audiovisual warning means 26 can be a bell, a bell and light, or any combination or any type of sound-producing and sight-producing means. Either audio or visual warning means can be used separately without the other within the description and intent of this invention. It is foreseeable that the audiovisual means can be hard-wired to an outside transmitter such that beeper, a phone, a speaker, lights, radio signals and other selections of warning devices would be actuated within the meaning of audiovisual warning as applied in this invention.

The invention can include a liquid crystal display(LCD) attached to or be part of the overall unit. The LCD depicted in FIG. 2 can show a readout of information from the device such as but not limited to elapsed time, pressure settings, and any other valve settings, and the like, as well give immediate or latent status data of measured parameters such as but not limited to actual pressure of fluid flowing through the tube, and the like, at any given time. The LCD display can include a transceiver(also depicted in FIG. 2) for allowing for remote actuation of the device and/or for sending device data to remote locales, such as but not limited to a nurse station, and the like. The transceiver can be either or both wireless, or hardwired, and the like, and use transmission modes including but not limited to radio frequency(rf), infrared red(IR), and the like. When combined with the LCD display, the warning system can send data to remote locales or display data when preselected thresholds are reached, or the LCD can be used without the warning system.

The brake coil 21 can be caused to release brake shoe 18 against brake resilient member 19 at the same time that the electromotive means 5 is caused to open the contractible member 1 and the audiovisual means 6 is actuated. This is illustrated variously In FIGS. 1,2,3,7,8 and 9.

Referring to FIG. 2, flow of fluid being regulated by this invention originates from a fluid source 21 and travels towards a fluid destination 28. When this Invention is being employed as a bladder cycler, the fluid source 27 is the bladder of a patient and the fluid destination 28 is a means for disposing of urine from the bladder. When this invention is being utilized as a programmable medication infusion system, the fluid source 27 is a medication reservoir and the fluid destination 28 is the blood stream or digestive tract of a patient. The main difference is in how it is used.

Functionally, the invention is substantially the same for bladder cycling as for inserting medication into the body of a patient. There are differences only in the types of transducers and the programming utilized. For implantation in bodies of patients, however, the invention can be designed and constructed much smaller and large control knobs would be replaced with minute control members.

Differences in relation to transducers would include an optional chemical transducer 29 or an optional electrical transducer 30 in place of or in addition to an optional pressure transducer with part numbers 22 through 25. An electrochemical transducer could be employed to combine the function of chemical transducer 29 and electrical transducer 38.

Details of particular chemical and electrical transducers are not included in this invention. There is a wide variety that can be employed. Typically, chemical transducers would be activated by and measure pH ion concentration for acidity or alkalinity, pH is measured in two basic ways: (1) colorimetrically and (2) electrometrically. For on-line controls such as this invention, standard glass electrodes could be employed electrometrically.

Electrical transducers could be variations of the standard Wheatstone Bridge. This could measure accurately such factors as electrical resistance, capacitance and inductance in conjunction with electrochemical, electromechanical and electro-optical sensors in the body fluid of patients. Electro-optical techniques can include a diode type light source and detector that can include a spectrometer, and the like.

Other factors that could be measured and utilized for actuation of transducers are flow rate, flow velocity, fluid viscosity, optical density, optical turbidity and optical color in relation to body fluid of patients.

For a variety of industrial and chemical applications of this invention, variations of the principles employed for measurement and transducer actuation in relation to body fluid can be employed for regulating flow of other types or fluid.

A tubing flow-control section 31 can be attachable to flexible tubing at either or both the outlet and inlet sides of the contractible member 1 and base member 2. Such a tubing flow-control section 31 can be selectively collapsible and less resistant to movement of the motive member 4. This would decrease power requirements and thus decrease the size and weight of the invention. It would also decrease accumulation of fluid in the control section 31 and thus also decrease conditions susceptible to buildup of contamination.

Resiliency of the flexible tube is important for many of the applications of this invention. Resiliency can be a tradeoff design factor with collapsibility for applications in which flow pressure is low enough for collapsibility to deter flow. Thus, flexibility is intended to include sufficient resiliency of tubes through which flow is regulated with this invention.

At either or both sides of the control section 31, there can be sealable valves 32 for taking out or putting in fluid separately from flow of fluid through the flexible tube 3. Depending on the application of the invention, these sealable valves 32 could be used to inject medication, a cultured substance or any modification or mix of the fluid at either the input or the outlet side of the valve. The sealable valves also could be used for sampling the fluid flowing through the control section 31. The sealable valves 32 also could be positions at which additional control transducers could be added.

The entire flexible tube 3 can be collapsible for some applications. For some applications, the entire length of the tube 3 would be no longer than illustrated relatively for the control section 31. Both for bladder cycling and for medication infusion, collapsible tubing may be preferable for the entire length of tubing employed.

When this invention is utilized for industrial or consumer applications not associated with health-care, medical and hospital uses, it can be constructed in sizes to match any size of tubing or pressure conditions. It can range in size for three-foot-diameter flexible tubing flow-control sections 31 down to flow-control sections less than an eighth of an inch in diameter. It is significant also that fluid conveyances leading to and from this invention can be solid plumbing or other tubing and fluid conveyances when the tubing flow-control section 31 is appropriately flexible, resilient or collapsible.

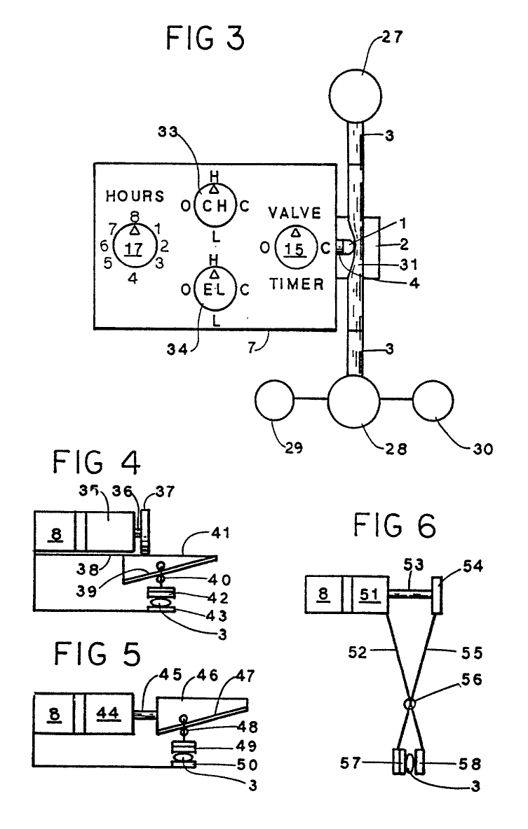

Referring to FIG. 3, an embodiment or this invention employing chemical and electrical transducers is illustrated from the top with chemical transducer knob 33 and electrical transducer knob 34 in place or the pressure knob 16. This form of the invention is primarily for medication infusion. A timer knob 17 and a regulator knob 15 are shown at opposite ends of the invention for continuity and clarity of description. The invention is also shown proportionately smaller for inference of its smaller construction for implantability in the body of patients. It could be made smaller yet by placing all four knobs parallel and at right angles to each other. The knobs can be made much smaller and the mechanisms employed could be miniaturized for implantation. Abbreviations rather than words for chemical, electrical, open, close, high and low are used for decreasing size.

Miniaturization could be aided by appropriate collapsibility and yet resilience of tubing, particularly the tubing flow-control section 31 for the reasons described above.

The base member 2 in FIG. 3 is shown narrower and shorter than in FIG. 2 because it does not include the pressure transducer at that position. Also in FIG. 3, the chemical transducer 29 and the electrical transducer 30 are shown for ease of illustration at opposite sides of a fluid destination 28. This is a partially schematic representation of separate transistors in relation to minute fluid disposition conditions in the body of a patient.

Illustrated schematically in FIGS. 4, 5 and 6 are alternative components that are foreseeable as mechanisms included within the basic description or this invention. Portions of these schematics not illustrated or described are assumed to be well-known to those skilled in the art to which they apply.

Included in FIG. 4 are geared rotational motor 35, preferably a reversible step motor, which rotates a shaft 36 to which a gear wheel 37 is attached. The gear wheel 37 can be “worm” geared for actuation of geared motive member 38 linearly to the axis of the shaft 36. Attached to the geared motive member 38 is an inclined cam 39 which actuates dual cam-follower 40 at right angles to the axis of rotational motor 35 and actuates a rotational-motor motive member 41 with rotational-motor contractible member 42 attached. The contractible member 42 is caused to travel in directions towards and away from rotational-motor base member 43 by opposite-directional rotation of rotational motor 35.

Opposite sides of flexible tube 3 are caused thereby to be pressed together or allowed to open selectively by fluid pressure within the tube and or by resiliency of the tube for achieving regulation or flow through the flexible tube.

Features of the invention not illustrated in relation to the schematic representations are assumed to be similar to those described in relation to other illustrations and related descriptions.

Illustrated in FIG. 5, geared linear motor 44, preferably a step motor actuates linearly in both directions a linear-motor shaft 45 to which a direct-drive motive member 46 is attached. On the direct-drive motive member 46 is linear-motor inclined cam 47 which actuates linear-motor dual cam follower 48. Attached to linear-motor dual cam follower 48 is linear-motor contractible member 49 which is actuated in both directions selectively towards and away from linear-motor base member 50 by opposite-directional linear travel of linear-motor shaft 45.

Illustrated in FIG. 6, a levered linear motor 51 is swivelably attached to inside lever arm 52. Levered linear-motor shaft 53 with attachment member 54 is swivelably attached to outside lever arm 55. Lever arms 52 and 55 are swivelably attached to fulcrum 56. Selectively opposite-directional linear travel of levered linear-motor shaft 53 causes levered linear-motor contractible member 57 to travel selectively towards and away from levered linear-motor base member 58.

Referring to FIG. 7, a flow diagram of a bladder-cycler embodiment of this invention is illustrated with the components indicated by words. Fluid flows from a “BLADDER” to a “VALVE” and then to a “DISPOSITION COUPLING.” At the valve, alternative programs of either a timer, a pressure transducer or an override are selected for fluid flow to reach a disposition point at the disposition coupling. Programmed pressure operation of the valve is selected by rotating the regulator knob 15 in FIG. 2 to “SENSOR.” Programming can be set for a relatively high or low pressure by rotating the “PRESSURE” knob 16 to “HIGH” and “LOW” settings respectively as illustrated further in FIG. 2. The “BRAKE” will maintain the valve in a closed position with contractible member 1 pressing the sides of flexible tube 3 against the base member 2 until the designated pressure is reached. When the pressure-sensing member 22 is activated as programmed, there will be an audiovisual warning as designed and programmed, the brake shoe 18 will release the motive member 4 and fluid will be allowed to flow through the flexible tube.

If, in addition, the regulator knob 15, illustrated in FIG. 2, is rotated to one side or the other of 12 O'clock position, the proportion of full open condition of the valve will be determined by the relative rotation or regulator knob 15 towards open and shut respectively. When a programmed low pressure has been reached, the valve will again close and remain in a closed position by action of brake shoe 18 until a programmed higher pressure is reached in the tubing flow-control section 31.

Timed opening is programmed by rotating the regulator knob 15 in FIG. 2 to “TIMER” at 6 O'clock. Then the time period between openings is selected by rotating the timer knob 17 to the indicated hours and portions of hours clockwise on the timer dial. At the time intervals programmed accordingly, the valve will open by travel of contractible member 1 away from the base member 2 and the flexible tube 3. When the valve opens, the audiovisual warning will be activated and the brake will be released and reset with the valve in open condition. When flow has stopped as indicated by absence of pressure in the tube against the pressure-sensing member 22, the brake will be released from open condition, the valve will be shut and the brake will be set again to maintain closed condition without expenditure of electrical energy for continued braking action.

Referring to FIG. 8, a flow diagram of the medication-insertion embodiment in FIG. 3 is illustrated with components indicated by descriptive words. Fluid flows from a “MEDICATION RESERVOIR” to a “VALVE” and then to an “INSERTION COUPLING.” At the valve in the medication-infusion application, alternative programs of either a timer, chemical transducer, electrical transducer or manual override are selected for fluid flow to reach a destination at the insertion coupling. To select programming for either or both of the chemical and electrical transducers, the regulator knob 15 is rotated to where the arrow point to “VALVE.”

The knobs are marked with “CH” for the chemical knob and “EL” for the electrical knob. Either or both knobs are programmed by first rotation to a position clockwise or counterclockwise from “H” for high. If the knobs are rotated to any position of rotation between “H” and “L” at the half-circle side marked “C” for closed, the valve will remain open until a relatively high or low chemical or electrical condition being programmed for is reached according to the relative rotation of the arrow between “H” and “L.” When the selective condition is reached, the valve will close.

If the knobs are rotated at any position of rotation between “H” and “L” at the half-circle side of the dial marked “O” for open, the valve will remain closed until a setting for opening is reached according to the position of rotation of the arrow in relation to “H” and “L.”

Both knobs must be on the “O” or the “C” side of the dial circles if both electrical and chemical transducers are being programmed. Otherwise, one will cancel the other out because there is only one valve for both. This is consonant with programming regimes for medication Infusion because generally the valve should stay either open or closed until either or both chemical and electrical conditions occur.

Timed opening or closing of the valve is selected the same for the FIG. 3 embodiment as for the FIG. 2 embodiment.

The FIG. 8 flow diagram indicates “ARIABLE OPEN-SHUT,”“AUDIOVISUAL WARNING” and “BRAKE” for the transducers and for the timer selections. Variable open and shut are selected as described above by rotation of the arrow on each knob to the relative position of chemical and electrical conditions or of open or closed conditions that are programmed with rotation of the knobs. Actuation of the brake and the audiovisual warning can be automatically simultaneous as programmed the same as described above for FIG. 2 in relation bladder cycling.

Other uses of this invention for industrial and consumer uses can employ similar programming and control parameters, depending on the particular applications.

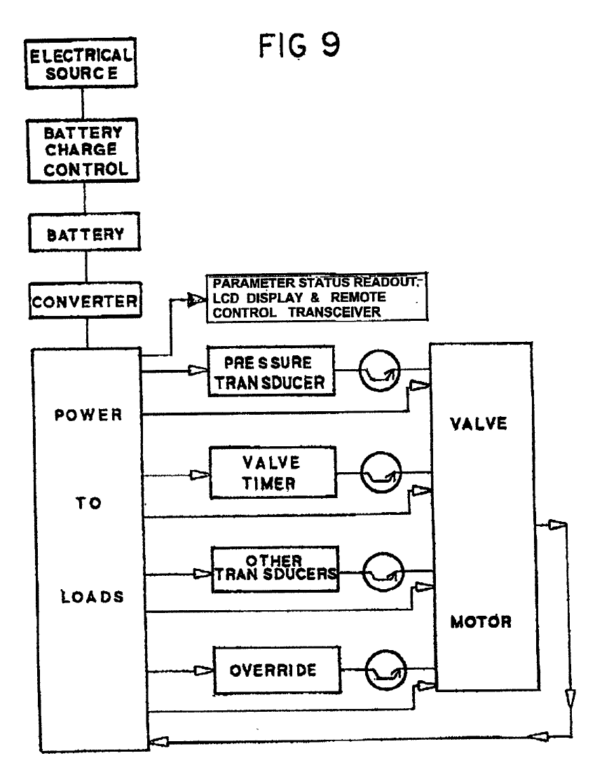

Referring to FIG. 9, an electrical circuit diagram is illustrated with descriptive words and symbols. From an “ELECTRICAL SOURCE,” current flows to a “BATTERY CHARGE CONTROL” and then to a “CONVERTER.”

The battery charge-control unit is separate from the battery to prevent potential electrical hazards. Shock-level electrical current is prevented from reaching a patient when a low-voltage batter is being employed as described above. A converter is employed to convert various levels of current to the particular applications for transducer use and for valve-motor operation. This division of current is indicated functionally in the diagram by “POWER TO LOADS.”

Current flows to a “PRESSURE TRANSDUCER,” as indicated by the arrow, and then to switching mechanisms for the “VALVE MOTOR.” Current flows separately to the valve motor because a relatively greater amount of current is required to operate the valve motor than the transducer mechanisms and switches. Transducer switches are indicated by the circled arrows. At the same time current flows to the valve motor, current flows also to the brake and warning devices as outlined in the flow diagrams, FIGS. 7 and 8. Electrical power to operate the brake and warning devices can be in line with flow of current to the valve motor. Electrical current to operate transducers which actuate the brake and warning mechanisms can be in line with the current to the pressure transducer

Current flows to a “VALVE TIMER,” to “OTHER TRANSDUCERS” and to an “OVERRIDE” in the same manner as to the pressure transducer. Other transducers can be any type or combination of types of transducers for any application of this device. Current flows separately to the “VALVE MOTOR.” Activation switches can be operated in line with the timer, transducers and override while mechanisms associated with the valve motor can be operated by current in line with the valve motor.

Current returns to the converter and the battery at the point where power is transmitted to loads associated with the mechanisms employed.

Current can also flow to power an LCD display and/or a transceiver which was described in reference to FIG. 2.

A wide variation of many additional combinations of the components comprising this device are foreseeable.

Although the invention has been described for use with bladder drainage and medication infusion applications, the invention can include other uses such as but not limited to industrial and consumer applications and the like. Such applications can include but not be limited to kidney dialysis, heart catheters, and neutral or cranial stents and the like. Additional medical areas for specific computer-chip controlled flow regulation units can also be used.

While the invention has been described, disclosed, illustrated and shown in various terms of certain embodiments or modifications which it has presumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

I claim:

1. A method of controlling fluid flow through tubes, comprising the steps of:
clamping a continuous longitudinal pliable tube to a side of a measuring unit;
sensing fluid flowing through the continuous longitudinal tube by sensing an exterior side wall portion of the tube; and
regulating the fluid flowing through the tube by exerting pressure on another exterior side wall portion of the tube to form an opening and closing valve, wherein the steps of clamping, sensing and regulating the fluid occur without removing portions of fluid from the fluid flow pathway passing through the tube.

2. The method of claim 1, further comprising the steps of:
connecting an upper end of the tube to a bladder; and
connecting a lower end of the tube to a disposition coupling, wherein the fluid includes urine.

3. The method of claim 1, further comprising the steps of:
connecting an upper end of the tube to a medication reservoir; and
connecting a lower end of the tube to a body insertion coupling, wherein the fluid includes medications.

4. The method of claim 1, further comprising the step of at least one of:
emitting a warning alarm when a selected pressure threshold has been sensed in the tube, and
displaying status information on the fluid flow through the tube.

5. The method of claim 1, further comprising the step of:
programmably opening and closing the valve with a timer.

6. The method of claim 1, further comprising the step of:
programmably opening and closing the valve with a selective pressure threshold.

7. The method of claim 1, wherein the step of sensing the fluid flowing includes the step of:
sensing pressure on the exterior side wall portion of the tube.

8. The method of claim 1, wherein the step of sensing the fluid flowing includes the step of:
chemically sensing the fluid through the exterior side wall portion of the tube.

9. The method of claim 1, wherein the step of sensing the fluid flowing includes the step of:
electrometrically sensing the fluid through the exterior side wall portion of the tube.

10. The method of claim 1, wherein the step of sensing the fluid flowing includes the step of:
optically sensing the fluid through the exterior side wall portion of the tube.

11. A programmable flexible-tube flow regulator, comprising:
means for clamping an exterior side portion of a continuous longitudinal flexible tube to a measuring unit;
means for sensing fluid flowing through the continuous longitudinal portion of the tube by sensing an exterior side wall portion of the tube in the unit; and
means for regulating fluid flowing through the tube by exerting pressure on another exterior side wall portion of the tube to form an opening and closing valve that controls the fluid flowing therethrough, wherein the clamping means, the sensing means and the regulating means occur without removing any portions of the fluid flowing through the tube.

12. The programmable regulator of claim 11, further comprising:
a bladder source connected to an upper end of the tube; and
a disposition coupling connected to a lower end of the tube.

13. The programmable regulator of claim 11, further comprising:
a medication reservoir connected to an upper end of the tube; and
a body insertion coupling connected to a lower end of the tube.

14. The programmable regulator of claim 11, further comprising at least one of:
means for emitting a warning alarm when a selected pressure threshold has been sensed in the tube, and
means for displaying status information on fluid flowing through the tube.

15. The programmable regulator of claim 11, further comprising:
a timer means for programmably opening and closing the valve.

16. The programmable regulator of claim 11, further comprising:
a pressure adjustment means selectively programming a selective pressure threshold for the opening and the closing of the valve.

17. The programmable regulator of claim 11, wherein the sensing means includes:
means for sensing pressure on the exterior side wall portion of the tube.

18. The programmable regulator of claim 11, wherein the sensing means includes:
means for chemically sensing the fluid through the exterior side wall portion of the tube.

19. The programmable regulator of claim 11, wherein the sensing means includes:
means for electrometrically sensing the fluid through the exterior side wall portion of the tube.

20. The programmable regulator of claim 11, wherein the sensing means includes:
means for optically sensing the fluid through the exterior side wall portion of the tube.
